(19) United States
(12) Patent Application Publication
(10) Pub. No.: US 2015/0142088 A1
(43) Pub. Date: May 21, 2015

(54) FEMALE UNDERGARMENT WITH HEATING COMPONENT

(71) Applicant: LESLIE E. RIVA GODOY, MAPLE (CA)

(72) Inventor: LESLIE E. RIVA GODOY, MAPLE (CA)

(21) Appl. No.: 14/542,121

(22) Filed: Nov. 14, 2014

Related U.S. Application Data
(60) Provisional application No. 61/904,650, filed on Nov. 15, 2013.

(51) Int. Cl. A61F 7/03

(52) U.S. Cl. CPC A61F 7/03; A61F 2007/0022
USPC 607/108; 607/112

(57) ABSTRACT

A device and method is taught for providing thermo therapeutic relief through the positioning of a heating element at a predetermined position in association with a garment to support and sustain the heat generated by the heating element in a designated area. The invention more particularly relates to a method and a device for treating menstrual cramps by application of thermo therapeutic relief to specific areas associated with a woman's abdomen. In addition a kit is taught for providing a particular type of undergarment and reusable heating pad for use therein, effective for relieving the discomfort of menstrual cramps.

### CROSS REFERENCE TO RELATED APPLICATIONS

This application claims benefit of priority to U.S. Provisional Patent Application Ser. No. 61/904,650, filed Nov. 15, 2013, the contents of which is incorporated herein by reference in its entirety.

### FIELD OF THE INVENTION

The present invention generally relates to a method and device for providing thermo therapeutic relief through the positioning of a heating element at a predetermined position in association with a garment to support and sustain the heat generated by said element in a designated area. The invention more particularly relates to a method and a device for treating menstrual cramps by application of thermo therapeutic relief to specific areas associated with a woman's abdomen.

### BACKGROUND OF THE INVENTION

Many women experience pain and discomfort during their menstrual cycle. While some women experience only moderate pain and light cramping during their monthly period, others suffer much more serious complications. In particular, extreme lower back pain and excruciating abdominal cramps go hand in hand with a monthly period for many women. This pain can be so debilitating that many women who suffer severe menstrual symptoms are forced to miss days of work, school or even planned social engagements.

Menstrual cramping (dysmenorrheal) is a medical condition characterized by severe uterine pain during menstruation. The condition is often attributed to contractions of the uterus as it expels unneeded contents and also the passage of clotted blood through the cervix. The pain associated with menstrual cramping is thought to result when the uterine muscles contract too strenuously resulting in severe discomfort around the abdomen, back and often the legs.

Doctors will sometimes recommend the application of heat to sooth the symptoms and relieve tenderness. The problem, however, is that most heat sources, such as heating pads, do not fit well around the abdominal region and therefore provide only partial relief. Moreover, heating pads and the like limit movement of the patient to the length of the electrical cords.

The prior art teaches the use of heat for the treatment of infections, muscular aches and different diseases, such as fibromyalgia and arthritic conditions, as well as menstrual cramping. Prior art heating means include heat baths and different types of heating pads and the like. Such known heat pads often have the disadvantage that they are ungainly and not suitable to be carried against the body, if the patient wants to move. The freedom of movement of the patients is therefore very restricted and this makes the possibilities of the treatment of patients during a longer time period more difficult. A common type of heating pad is intended to be placed in a bed and presupposes that the patient is lying down during the treatment.

While articles utilized to provide thermo therapeutic heat relief do exist, none have been heretofore intertwined with a mid thigh/high waisted undergarment for pain relief for menstrual cramps/discomfort.

### DESCRIPTION OF THE PRIOR ART

U.S. Pat. No. 4,077,390 to Stanley, et al. is directed toward a heat pack made by enclosing supercoolable aqueous sodium acetate solution together with a metallic activator strip in a sealed, flexible container. The activator strip is a flexible metal strip having one or more fissures or slits extending therethrough. To prepare the heat pack for activation, its contents are first heated to a temperature above the melting point of sodium acetate to completely melt it. Thereafter, the sodium acetate solution is supercooled. Activation or crystallization of the sodium acetate (with evolution of heat) is produced by bending the activator strip.

U.S. Pat. No. 4,628,930 to Williams is directed toward a girdle for use in preventing the discomfort of cramps due to a menstrual period. The girdle has a cotton cloth panty of a type having a front portion, a rear portion and interconnecting side portions. The panty has a continuous elastic material around the top thereof and has a battery holding compartment attached to the top to a central front portion thereof. An electrical resistance heating structure is attached to the outside of the cotton panty front portion and extends from the crotch portion thereof upwardly and outwardly in a V-shaped configuration to where the battery holding compartment is attached to the elastic. An electric battery is removably disposed within the battery holding compartment. A V-shaped basket weave cloth structure having a higher R-value than that of the cotton cloth of which the front portion is constructed is disposed over the front of the electrical heating structure for causing heat from the electrical heating structure to tend to flow through the cotton front panel towards the person wearing the girdle rather than through the front basket weave cloth whereby such person wearing the girdle will have that part of her anatomy which tends to cramp during menstrual periods heated during a menstrual period.

U.S. Pat. No. 4,753,241 to Brannigan, et al discloses a therapeutic device which includes a first flexible layer having a plurality of interconnected raised chambers and passageways. The upper surface of a second flexible layer is positioned opposite of the lower surface of the first flexible layer. The periphery of the overlying layers is heat sealed to form a plurality of chambers spaced from each other and connected by the passageways. A thermal responsive medium is introduced through the passageways into the chambers. Initially, the chambers are not completely filled. Thereafter the overlying layers are further heat sealed to remove the passageways between the chambers. Consequently, the liquid that was present in the passageways is evacuated into the chambers, filling the chambers which are individually sealed from one another. For heat treatment application a third flexible layer is sealingly engaged to the lower surface of the second flexible layer and is wetted with an aqueous solution. The heated therapeutic device is then applied to the desired body portion with the cells of the flexible insulating member positioned against the body portion to be treated. The flexible nature of the therapeutic device permits the device to be compressively held in close conformity to the body portion to enhance the rate of transfer of hot or cold temperature to the effected body portion.

U.S. Pat. No. 5,050,595 to Krafft describes a women's therapeutic support garment comprising a pair of breast supporting cups each of which is formed with an inner and an outer panel defining there between one of two cupped shaped pockets. A cupped shaped, thermal gel pack is placed in each pocket and has a central opening for accommodating the women's nipple. A pair of side panels are connected to the breast supporting cups, the side panels being dimensioned and configured to encircle the wearer and hold the breast supporting cups in place with the gel packs surrounding the women's breasts. The heat from each gel pack serves to reduce swelling and tenderness of the breast tissues during the premenstrual period, pregnancy or the post-partum period.

U.S. Pat. No. 6,235,049 to Nazerian is directed toward a device for a heat treatment of a body part, comprising a heating element and means for holding the heating element against the body part to be heat-treated. The heat element comprises a plurality of separate heating cells and is flexible in such a manner that it may be adapted to abut said body part.

U.S. Pat. No. 7,135,036 to Yue describes a heating pad which includes an envelope containing a heating unit and a phase change material which encloses the heating unit. The heating unit includes a metal plate and a resistance heating element attached to the metal plate. Flexible strips are disposed within the envelope by attachment to the envelope, and the metal plate is positioned within the envelope through the strips. A pair of electrodes are connected to the heating element and extend outwardly of the envelope. The phase change material can undergo a phase change process through the heating unit so that no external heat source is needed to heat the phase change material.

U.S. Pat. No. 7,824,437 to Saunders teaches a portable electronic device for reducing periodic cramping about an abdominal cavity of a user during a menstrual cycle which includes an elastic panty garment, a controller removably attached directly to an outer surface of the panty garment and a plurality of electro-mechanical transducers generating output vibrations, and a plurality of heatable magnetic straps directly coupled to the outer surface of the panty garment. The heatable magnetic straps extend along anterior and posterior faces of the panty garment and are simultaneously actuated to a desired temperature level while the transducers are actuated to an on position. The heatable magnetic straps are freely movable along the outer surface of the panty garment while the transducers are nested within respective pouches.

United States Patent Application 20100023099 to Hidaka et al. describes a heat generating device which includes an air-permeable first side located proximal to a wearer's body, a second side located distal to a wearer's body, and a heat generating member between the two sides. The second side includes an adhesive layer for attaching the device to a garment. The first side is formed of a nonwoven fabric having an uneven surface topography. The nonwoven fabric includes a first fiber layer, inclusive of a first surface of the fabric, and a second fiber layer, inclusive of a second surface of the fabric, partly bonded to each other and including a large number of protrusions and recesses on the side of the first fiber layer. The first surface is used as the first side.

United States Patent Application 20110041839 to Lee et al. teaches a compression undergarment to relieve the symptoms of menstrual cramping that comprises a first and second pad both capable of conforming to a female user's hips. Each pad is tapered having two part construction. Such construction may include a pliable exterior panel of resilient material and an interior panel made of compressible material. The second component of the apparatus is a compression undergarment having a first and corresponding second annular sleeve. Such compression undergarment includes a front side, back side, first leg portion and second leg portion, such that positioned above the first leg portion and second leg portion is a top portion which connects both leg portions. The compression undergarment also includes a first annular sleeve capable of receiving the first pad and a second annular sleeve capable of receiving the second pad. The third component is a compression band that fits around both pads and the compression undergarment.

United States Patent Application 20110231979 to Nilforushan et al. is directed toward human clothing which has inner and outer flexible layers that define a space sufficiently large to position a thermal transfer element in any of multiple, non-overlapping positions. The space and the thermal transfer element preferably have cooperating sides of a hook-and-loop attachment. All suitable items of clothing are contemplated, but especially a shirt, jacket or pants that appear to others as a substantially normal garment. Each space has one or more openings, in either the inner or outer layers, and preferably at the shoulders or sides of the torso, or at the pocket areas of pants. The spaces are preferably large, not only relative to the size of a corresponding thermal transfer element, but also to the size of the garment. Thermal transfer elements are preferably disposed in pouches having a thermal insulation on that side, and have only a very thin, thermally transmissive, fabric on the other side.

In summary, in one embodiment, the prior art teaches devices comprised of shorts formed of one fabric (elastic material) with a multitude of external straps, pouches, control like panels etc. In another embodiment the art is directed toward a girdle with cotton cloth with interconnection portions with a heating structure positioned externally, wherein as it relates to the heating element, this product primarily utilizes a battery powered mechanism to heat the girdle. In yet another embodiment, the prior art requires an air-permeable component as well as a generating member component with an adhesive layer. In a still further embodiment an annular sleeve is required while utilizing a compression type garment. In a still further embodiment transfer heat elements are utilized including singular and multiple plastic gel pads with hook and loop attachments with knitted or woven fabrics facing the thermal transfer element with suitable closure components.

What the prior art fails to teach is a female undergarment constructed and arranged to position a heat pack in order to alleviate menstrual cramping in either the abdominal or lumbar regions. What the art further fails to teach is a self-renewing heat pack wherein the heat-generating material within the heat pack is compartmentalized in segmented areas of the overall heat pack in order to ensure that the heated material does not shift away from the affected areas during movement, or when the wearer lies down.

### SUMMARY OF THE INVENTION

The present invention provides an innovative method in which thermo therapeutic relief is intertwined with an undergarment thus allowing mobility and relief simultaneously.

Contrary to the teachings of the prior art embodiments, the present invention contains a singular heating element containing acetic acid sodium salt trihydrate (food grade) and water in a bag or pouch with a metal disc, that does not require the use of any electromagnetic type devices.

Accordingly, in an embodiment, the instant invention provides a thermo therapeutic device whose design emulates that of an undergarment, specifically a mid-thigh, high-waisted garment with an internal pocket formed of different fabrics providing effectiveness, comfort and relief simultaneously. Unlike the prior art devices, it does not have electro-mechanical transducers, magnetic straps, heatable magnetic straps, electrical massaging mechanisms, controllers, electrical thermal pads, batteries etc.

In an embodiment, the instant invention illustrates a thermo therapeutic device that places the activated heating device into the garment's pocket, thus avoiding contact with the end user's undergarment. The present invention does not utilize moist heating devices nor heat transfer methods that generate steam. Rather, the invention utilizes a small metal component that once clicked, crystallizes the invention's exothermic mechanism, which utilizes direct heat generated via crystallization of the components of the heating pad, so as to reach a set temperature of about 54° C. via a molecular chain-like reactive method.

In an embodiment, the invention illustrates a garment containing a built in pocket for the designated uterine area without attachments or closures, which is specifically designed to provide thermo therapeutic relief of the uterine area with a singular pocket and heating element. Its heating element is reset via placing the heating pad in boiling water for approximately seven minutes and once liquefied it is reset as specified in an earlier statement.

In an embodiment, the present invention provides a thermo therapeutic device which utilizes a thermo therapeutic device constructed and arranged in the form of a pouch containing sodium acetate trihydrate in a plurality of segmented compartments within the pouch, the segmented compartments being effective to maintain the sodium acetate within the segments from freely moving within the pouch in response to movement, thereby enabling an even heating effect to be maintained during movement or changes in position, e.g. lying down versus standing up.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A-1C shows a stepwise illustration depicting activation (FIG. 1A), normal use (FIG. 1B, and recharging (FIG. 1C) of the heating pad insert;

FIG. 2 illustrates a the heating element containing the segments and perforations for providing stable and even heat transfer;

FIG. 3A illustrates a frontal view of the undergarment;

FIG. 3B illustrates a posterior view of the undergarment;

FIG. 4A illustrates a frontal perspective view of the undergarment containing a uterine pouch for the heating element;

FIG. 4B illustrates a posterior perspective view of the undergarment;

### DETAILED DESCRIPTION OF THE INVENTION

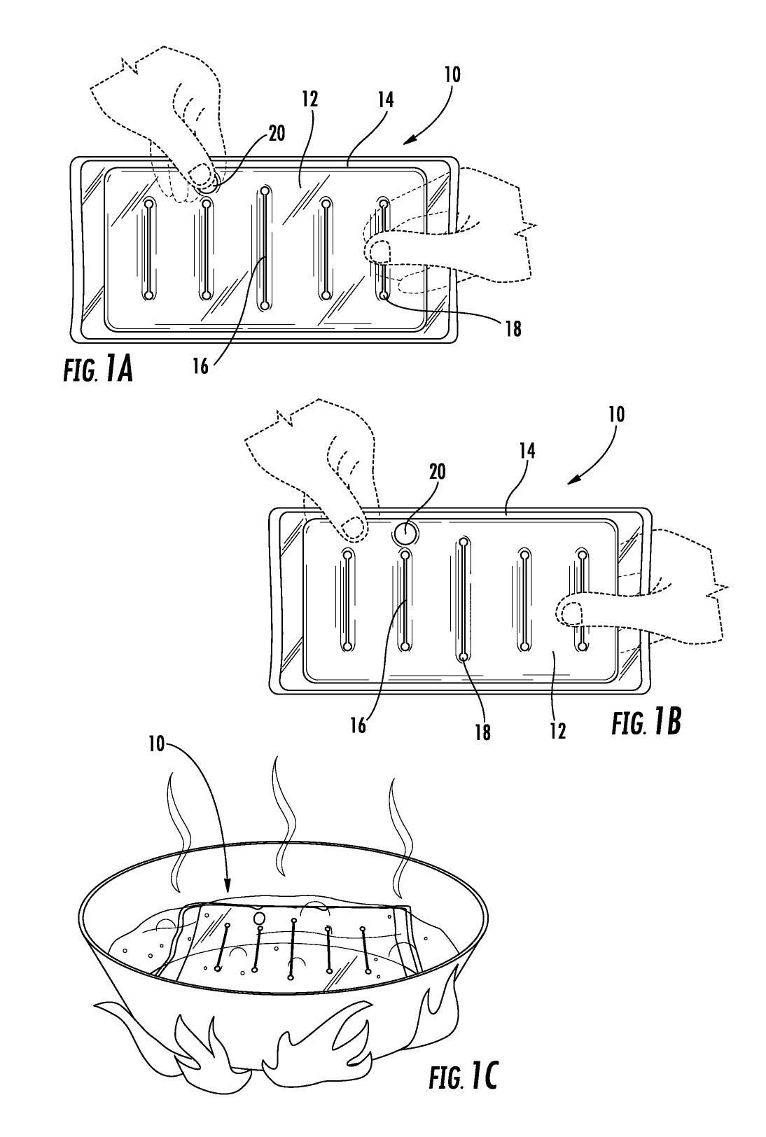

With reference to FIGS. 1A-1C, the heating element mechanism of action relies upon a chemical reaction, specifically a crystallization initiated by the presence of the metal disc, which when flexed initiates the crystallization process.

As illustrated in FIG. 1A, the reusable heating element mechanism or heat pack 10, comprises a solution containing sodium acetate trihydrate crystals 12 which are encased within a polymeric pouch 14. The pouch 14 has a number of welds 16 and perforations 18. The welds 16 act to keep the sodium acetate trihydrate 12 from shifting to a great degree during movement of the user and/or during changes of position, e.g. from a standing to a supine position. Upon flexing of an activation disk 20 the sodium acetate trihydrate crystals 12 melt at about 137.12° F./58.4° C., dissolving in their water of crystallization. By pressing on the metal disc 20 within the heating pad, a nucleation centre is formed, causing the solution to crystallize back into solid sodium acetate trihydrate. The bond-forming process of crystallization is exothermic. The latent heat of fusion is about 264-289 kJ/kg. Unlike some types of heat packs, such as those dependent upon irreversible chemical reactions, a sodium acetate heat pack can be easily reused. FIG. 1B is illustrative of an activated heating mechanism 10 in its active heating phase. As illustrated in FIG. 1C, after the process is exhausted, the device may be reliquified by immersing the pack 10 in boiling water for a few minutes. When the crystals 12 are heated past the melting point and subsequently allowed to cool, the aqueous solution becomes supersaturated. This solution is capable of cooling to room temperature without forming crystals.

Referring to FIG. 2, a preferred embodiment for the heating element mechanism or heat pack 10 (alternatively referred to as a heating pad) is a perforated embodiment as herein illustrated. The heat pack 10 encases sodium acetate trihydrate crystals 12. The pouch 14 has a number of welds 16 and perforations 18. The welds 16 act to keep the sodium acetate trihydrate 12 from shifting to a great degree during movement of the user and/or changes of position, e.g. from a standing to a supine position. This enables an even heating profile to be maintained. The perforations 18 permit the evaporation of moisture therethrough, along with circulation of air to allow flow of moisture away from the body and assist in providing even heating to the affected area. Upon flexing of an activation disk 20 the exothermic reaction of the sodium acetate trihydrate crystals is initiated.

In an exemplary, albeit non-limiting embodiment, the heating element 10 contains an acetic acid sodium salt trihydrate (food grade) 12 and water in a bag 14 with a metal disc 20. To utilize the heating pad 10, the metal disc 20 must be flexed as the liquid creates a chain reaction causing the pad to crystallize. This crystallization occurs at a temperature of about 130° F./54° C. Once activated, the pad has available to it a predetermined amount of energy. It will use this energy to increase the temperature of the pad to approximate 130 degrees F./54 degree C. and use whatever is left to maintain that temperature. This ability to produce heat for a period of time is accomplished because all of the liquid does not crystallize immediately. Molecules that were not required to heat the pad to its maximum temperature will remain liquid, and crystallize as required, to maintain the maximum temperature. The pad 10 is fully activated in about 12 seconds and retains the target temperature for about four hours, and can be reused an unlimited number of times.

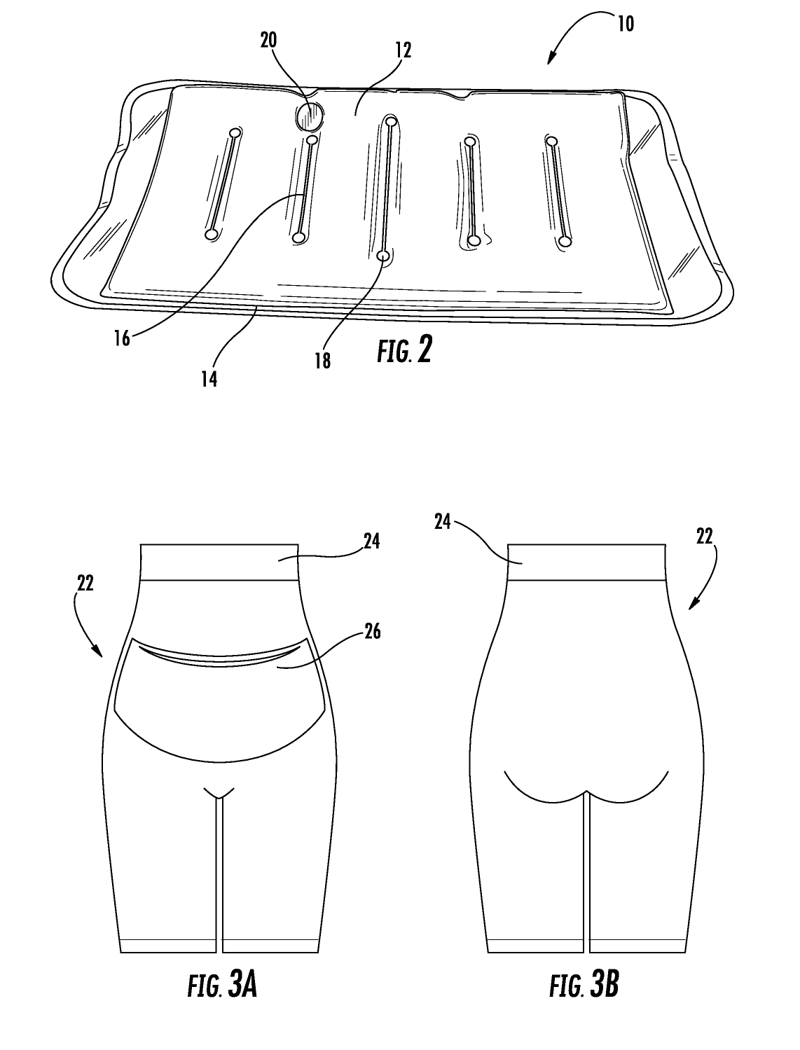

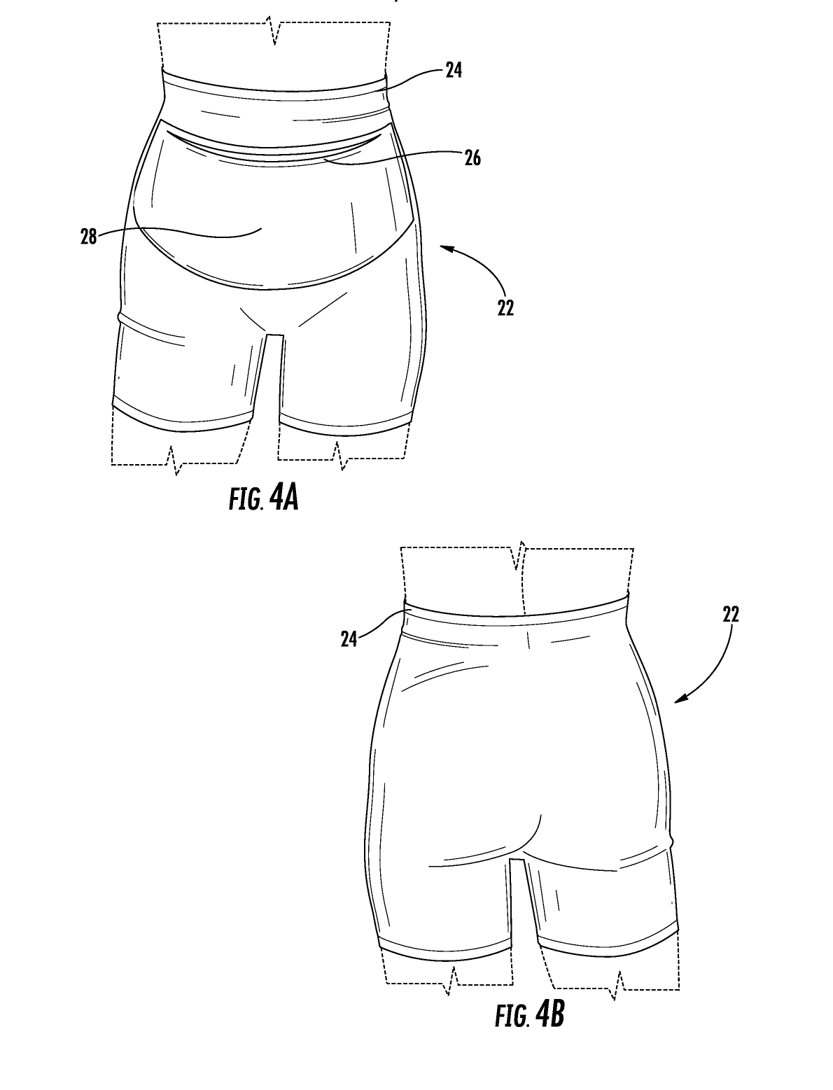

As illustrated in FIGS. 3A, 3B, 4A, and 4B, there is disclosed a fusion between wearability and pain relief through the combination of an undergarment with an internal pocket sustaining the thermo therapeutic element thus providing both mobility and pain relief simultaneously. In an exemplary, albeit non-limiting embodiment, the garment is formed from a 218 gr/m² knitted fabric, e.g. a single jersey knit, with elastomeric and rubber thread, which is dyed and non-textured, having a filament yarn count of about 100 d/192×20 D. The insert garment may be formed from a piece dyed, 182.44 gsm, tricot knit, with elastomeric and rubber thread dyed, filament, non-textured. Tricot is the name of a particular type of warp knit fabric, accomplished on a tricot knitting machine. Basically, in a tricot knit, two parallel lines of yarns are interloped, the loops being formed in roughly the warp direction. As compared to filling knits such as jersey, tricot knits are known for being dimensionally stable and not running.

In an alternative embodiment designed as a youth garment, the fabric is a nylon.

Now referring to FIG. 3A and 4A a frontal view of the present invention is illustrated. According to standard sizing methods within the garment industry, the undergarment 22 was created with the following sizes, XS/S being extra small or small, S/M being small or medium and L/XL being Large or Extra Large. Numeral 24 indicates the waist band or topmost element of the garment which also includes an elastic band therein equaling the height of the waist band. Numeral 26 indicates the opening within internal pocket 28 for receipt therein of the heating element 10. FIG. 4A illustrates a frontal perspective view of the undergarment containing a uterine pocket 28 for the heating element 10.

Now with reference to FIG. 3B and 4B, the posterior view of the undergarment 22 is described as it relates to the numeric allocations within the illustration. Numeral 24 indicates the waist band or topmost element of the garment which also includes an elastic band (not shown) equaling the height of the waist band 24.

In one embodiment, the invention is contemplated as being in the form of a kit including Instructions for Usage, one or more undergarments, and one or more heating pads.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A device for providing thermo therapeutic relief through the positioning of a heating element at a predetermined position in association with a garment to support and sustain the heat generated by said element in a designated area comprising:
an undergarment having an integral pocket constructed and arranged for receipt of a heating element;
a reusable heating element including a pouch encasing a volume of sodium acetate trihydrate, and a flexible metal disk, which initiates an exothermic reaction upon being flexed;
said pouch having a plurality of welds effective to keep the sodium acetate trihydrate from shifting during movement of the user thereby enabling even heating to be maintained;
said perforations constructed and arranged to permit evaporation of moisture along with circulation of air therethrough, to allow flow of moisture away from the body and assist in providing even heating to the affected area;
whereby, upon activation an even and prolonged heat, at a predetermined position, is provided to the user.

2. The device of claim 1, wherein the device is constructed and arranged for treating menstrual cramps by application of thermo therapeutic relief to specific areas associated with a woman's abdomen.

3. The device of claim 1 wherein the garment is formed from a jersey knit fabric having elastomeric and rubber threads.

4. The device of claim 1 wherein the integral pocket is formed from a tricot knit fabric having elastomeric and rubber threads.

5. The device of claim 2 wherein the garment is formed of nylon.
